(19) United States
(12) Patent Application Publication
(10) Pub. No.: US 2022/0211357 A1
(43) Pub. Date: Jul. 7, 2022

(54) SYSTEMS AND METHODS FOR PERFORMING TISSUE BIOPSY

(71) Applicant: OLYMPUS MEDICAL SYSTEMS CORPORATION, HACHIOJI-SHI (JP)

(72) Inventor: David A. Herrin, Seattle, WA (US)

(21) Appl. No.: 17/141,710

(22) Filed: Jan. 5, 2021

(51) Int. Cl. A61B 10/02; A61B 10/04

(52) U.S. Cl. CPC A61B 10/0283; A61B 10/04

(57) ABSTRACT

Various disclosed embodiments include illustrative apparatuses, systems, and methods for providing vacuum forces for tissue biopsies. An illustrative apparatus includes a pump housing having a distal end attachable to a medical device and a longitudinal axis, a piston slidably receivable within the pump housing, and a handle rotatably attachable to the pump housing and the piston. The piston is configured to create a seal between a distal portion of the pump housing and a proximal portion of the pump housing. The handle includes a portion that is configured to move in a direction different from the longitudinal axis of the pump housing.

### TECHNICAL FIELD

The present disclosure relates to suction devices used with medical devices.

### BACKGROUND

The statements in this section merely provide background information related to the present disclosure and may not constitute prior art.

In current biopsy procedures, such as in fine needle aspiration (FNA) biopsy, a vacuum syringe is used to provide a suction force in order to assist a needle in aspirating tissue. As shown in FIG. 1, a syringe, such as that produced by Vaclok™, is attached to a stopcock. The stopcock is then attached to a medical device handle, such as a needle handle, that may be received within a working channel port of an endoscope. This procedure requires multiple steps including opening the stopcock then drawing vacuum by pulling the piston of the syringe proximally. This procedure requires help from an additional operator because attaching the stopcock and the syringe as well as operating the syringe requires two hands to operate.

### BRIEF SUMMARY

Various disclosed embodiments include illustrative apparatus, systems, and methods for providing a vacuum force during tissue biopsies.

In an illustrative embodiment, an illustrative apparatus includes a pump housing having a distal end attachable to a medical device and a longitudinal axis, a piston slidably receivable within the pump housing, and a handle rotatably attachable to the housing and the piston. The piston is configured to create a seal between a distal portion of the pump housing and a proximal portion of the pump housing. The handle includes a portion that is configured to move in a direction different from the longitudinal axis of the pump housing.

In another illustrative embodiment, an illustrative system includes a medical device having a handle and an insertion portion. An apparatus includes a pump housing having a distal end attachable to the medical device and a longitudinal axis. A piston is slidably receivable within the pump housing, and the piston is configured to create a seal between a distal portion of the pump housing and a proximal portion of the pump housing. A handle is rotatably attachable to the housing, and the handle is configured to move in a direction different from the longitudinal axis of the pump housing.

In another illustrative embodiment, an illustrative process places a hand pump in a pre-activated state. The hand pump includes a piston, a piston housing that receives the piston therein, a fitting located at a distal end of the piston housing, and a handle that move the piston within the piston housing. The hand pump is placed in the activated state responsive to a distal end of a medical device being disposed adjacent to a target tissue and the fitting being coupled to the medical device.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

Illustrative embodiments are illustrated in referenced figures of the drawings. It is intended that the embodiments and figures disclosed herein are to be considered illustrative rather than restrictive.

FIG. 1 is a side view of a prior art tissue biopsy system.

FIG. 2 is a side view of an illustrative tissue biopsy system.

FIG. 3 is an isometric view of an illustrative vacuum device.

FIG. 4 is a partial x-ray view of the vacuum device of FIG. 3.

FIG. 5 is a partial cross-sectional view of the vacuum device of FIG. 3.

FIG. 6 is an x-ray view of an illustrative vacuum device in a first mode of operation.

FIG. 7 is an x-ray view of the illustrative vacuum of FIG. 6 in a second mode of operation.

FIG. 8 is a flowchart of a method performed by an illustrative tissue biopsy system.

FIG. 9 is an isometric view of an illustrative vacuum device.

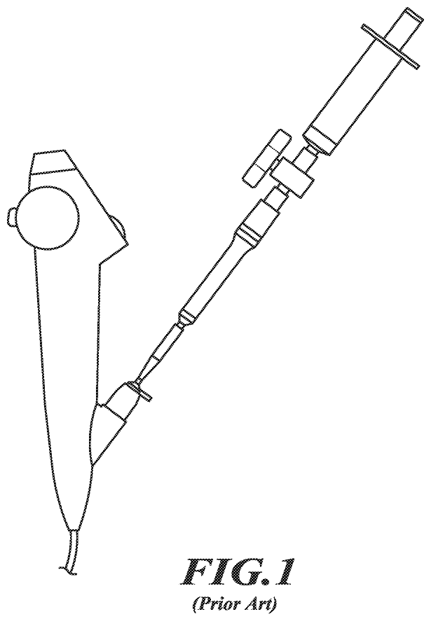

Like reference symbols in the various drawings generally indicate like elements.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Various disclosed embodiments include illustrative apparatuses, systems, and methods for producing continuous or near continuous vacuum while aspirating tissue.

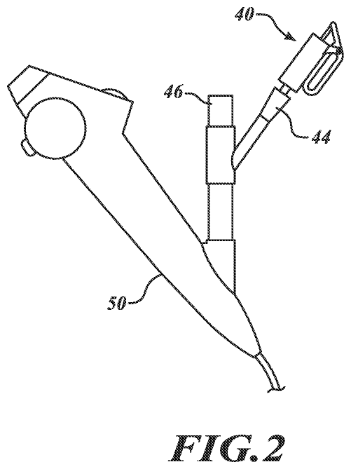

Referring now to FIG. 2 and given by way of overview, in various embodiments an illustrative system includes a scope 50, a multi-lumen catheter system 46, an aspirating device 44, and a vacuum-creating device 40.

It will be appreciated that the scope 50 may be any of a number of different types of scopes used in medical procedures, such as bronchoscopes, laparoscope, laryngoscope, etc. The scope 50 may include multi-use or single-use endoscopes. The multi-lumen catheter system 46 may be any device that includes two or more lumen located within an insertion tube attached to a handle device. The lumen of the multi-lumen catheter system 46 may be suitable for receiving imaging devices and the medical devices 44. The received imaging devices may include cameras or ultrasound devices, such as a radial ultrasound probe or comparable device. In various embodiments, the multi-lumen catheter system 46 may include a base section rotatably coupled to the scope 50 and a port slidably received by the base section. An exemplary multi-lumen catheter system 46 is shown and described in U.S. Provisional Patent Application No. 63/123,731, filed Dec. 10, 2020, the contents of which are hereby incorporated by reference.

In various embodiments, the insertion tube of the multi-lumen catheter system 46 is received within a working channel port of the scope 50. The multi-lumen catheter system 46 may be attached to the working channel port. The aspirating device 44 may include a flexible sheath attached to a first portion of a handle and an operational tool slidably received within the flexible sheath and attached to an actuator portion of the handle. The operational tool may include a tissue aspiration device, such as a flexible needle, a cytology brush or comparable devices. The operational tool may include a lumen that is in fluid communication with a corresponding lumen within the first and actuator portions of the handle.

In various embodiments, the vacuum-creating device 40 sealably attaches to the handle of the aspirating device 44, such that the vacuum-creating device 40 maintains fluid communication with the lumens of the operational tool and the first and actuator portions of the handle of the aspirating device 44.

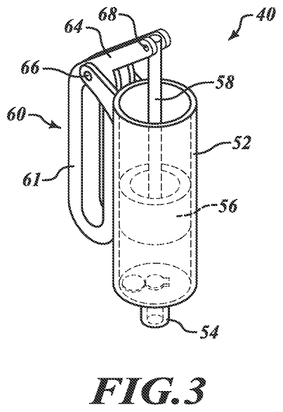

Referring additionally to FIG. 3, in various embodiments the vacuum-creating device 40 is a hand pump for drawing a vacuum. The hand pump includes a pump chamber 52 and an attachment device 54 (e.g., Luer fitting or other fitting depending upon the device that the vacuum-creating device 40 will attach thereto), a piston 56 slidably received within the pump chamber 52, a piston rod 58, and a handle 60. The piston rod 58 extends from a proximal side of the piston 56. The handle 60 includes a handle arm 64 and a grip 61. A first pivot pin 68 rotatably connects a proximal end of the piston rod 58 to a first end of the handle arm 64. A handle pivot mount 63 receives a second pivot pin 66 and is mounted to a proximal end of the pump chamber 52. The second pivot pin 66 is rotatably received within a connection between the handle arm 64 and the grip 61.

As will be described and shown in more detail below, as the grip 61 is pulled away from the pump housing 52, the grip 61 rotates about the second pivot pin 66, thus causing the handle arm 64 to apply a distal force to the piston rod 58. The piston rod 58 then moves the piston 56 distally within the pump chamber 52. As the grip 61 is moved toward the pump housing 52, the grip 61 rotates about the second pivot pin 66, thus causing the handle arm 64 to apply a proximal force to the piston rod 58. The piston rod 58 then moves the piston 56 proximally within the pump chamber 52.

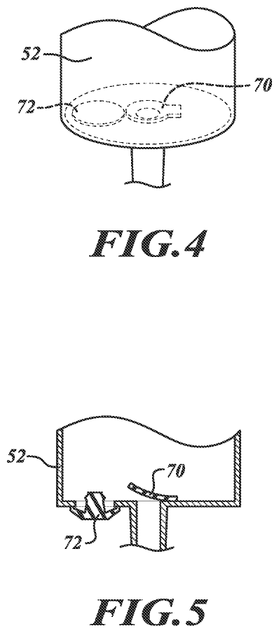

Referring additionally to FIGS. 4 and 5, in various embodiments a base or distal section of the pump chamber 52 includes an intake valve 70 and an exhaust valve 72. The intake valve 70 maintains a closed position between the pump chamber 52 and a lumen within the attachment device 54 when air pressure within the attachment device 54 is equal to or less than air pressure within the pump chamber 52. The intake valve 70 is in an opened position when the air pressure within the attachment device 54 is greater than the air pressure within the pump chamber 52. Examples of the intake valve 70 and the exhaust valve 72 include flapper valves, umbrella valves, duckbill valves, or comparable one-way valves.

The exhaust valve 72 is located between a wall of the pump housing 52 and an exterior environment of the pump housing 52. The exhaust valve 72 is in an opened position when the air pressure within the pump chamber 52 is greater than air pressure of the exterior environment of the pump housing 52. The exhaust valve 72 is in a closed position when the air pressure within the pump chamber 52 is less than the air pressure of the exterior environment of the pump housing 52.

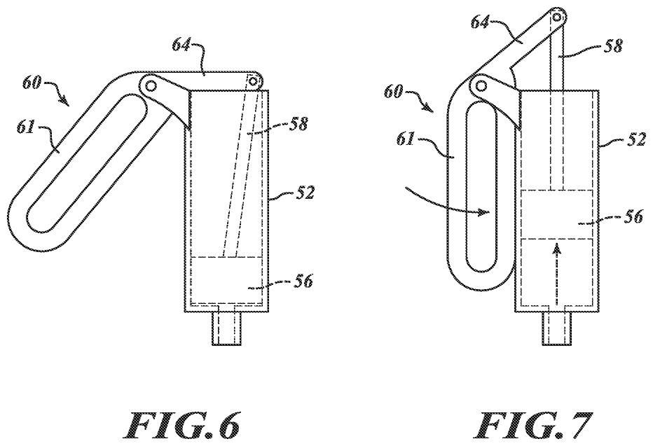

Referring additionally to FIGS. 6 and 7, in various embodiments when the vacuum-creating device 40 is actively being operated by the positions of the grip 61 and the piston 56 transition between those positions of FIGS. 6 and 7. In a pre-activation state of operation, the piston 56 is located at a distal position within the pump chamber 52 and the grip 61 is rotated about the second pivot pin 66 to a position away from a side of the pump chamber 52. In a post-activation state of operation, the grip 61 has been rotated in a counterclockwise motion about the second pivot pin 66 when looking at the vacuum-creating device 40 with the grip 61 positioned on the left side of the pump chamber 52. This motion of the piston 56 causes the exhaust valve 72 to close and the intake valve 70 to open due to the creation of lower air pressure in the pump chamber 52. The rotation of the grip 61 from the position of FIG. 6 to the position of FIG. 7 causes the handle arm 64 to apply a proximal force to the piston rod 58, thus forcing the piston 56 approximately within the pump chamber 52.

In various embodiments, repeated operation of the handle 60 as described above in FIGS. 6 and 7 results in more and more of a vacuum force to be created in the pump chamber 52. Air is evacuated from the pump chamber 52 when the piston 56 transitions from post-activation state of operation to the pre-activation state of operation.

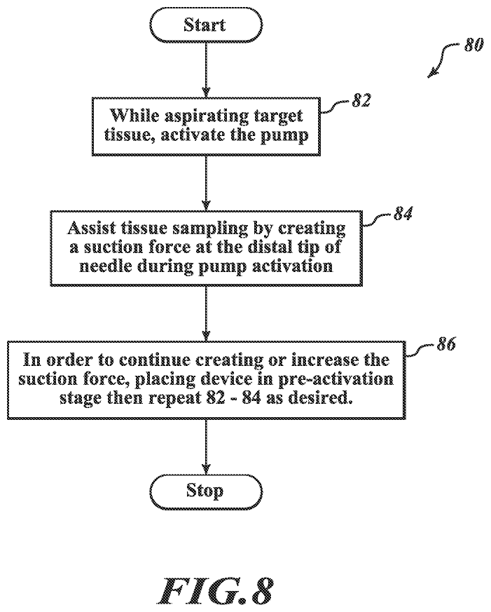

Referring additionally to FIG. 8, in various embodiments an illustrative process 80 is performed to enable tissue biopsies. At a block 82, while a user using a medical device is either in the process of actively aspirating target tissue or has a sampling portion of the medical device currently engaged with target tissue, the vacuum-creating device 40 is activated thus producing a proximal suction force at a distal end of the vacuum-creating device 40. At a block 84, sampling of the target tissue occurs at the distal end of the vacuum-creating device 40 based on the produced proximal suction force. At a block 86, if one desires to continue creating or increasing the suction force, the vacuum-creating device 40 is returned to a pre-activation stage then the steps described in blocks 82 and 84 are repeated. This may continue until the operator decides to stop the biopsy process and remove the medical device from the patient.

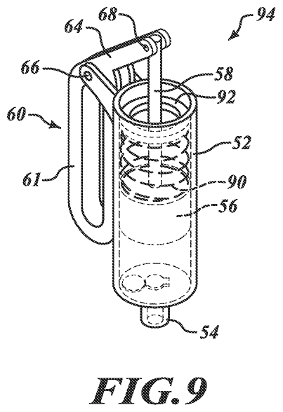

Referring additionally to FIG. 9, in various embodiments a vacuum-creating device 94 is a hand pump for drawing a vacuum. A stop feature 92 is located along an inner wall of the pump chamber 52 near a proximal end. A biasing device 90, such as a spring, is positioned within the pump chamber 52 between the piston 56 and the stop feature 92. The biasing device 90 acts upon the piston 56 to assist in expelling air from the pump chamber 52. In various embodiments, a biasing device may be located distally from the piston 56 for providing a force for generating a vacuum.

From the foregoing discussion and associated drawing figures, it will be appreciated that various embodiments have been disclosed and illustrated. To that end and without any implication of any limitation (which is not to be inferred), the following paragraphs set forth non-limiting summaries of various embodiments disclosed herein by way of example only and not of limitation:

A. An apparatus comprising: a pump housing having a distal end attachable to a medical device and a longitudinal axis; a piston slidably receivable within the pump housing, the piston being configured to create a seal between a distal portion of the pump housing and a proximal portion of the pump housing; and a handle rotatably attachable to the housing and the piston, the handle including a portion being configured to move in a direction different from the longitudinal axis of the pump housing.

B. The apparatus of A, wherein the pump handle including: a handle; a handle arm; and a pin pivot passage disposed between the handle and the handle arm, the apparatus further comprising: a fitting disposed at a distal end of the pump housing, the fitting attachable to a handle of a medical device; a piston rod couplable to a proximal side of the piston; a bracket mounted to an external surface of the pump housing at a proximal end of the pump housing; and a pivot pin couplable to the bracket and received within the pin pivot passage of the pump handle.

C. The apparatus of B, wherein the pump housing includes an intake valve disposed between the fitting and a distal end of the pump housing, the intake valve being configured to allow air to flow proximately between the distal end of the pump housing and the pump housing.

D. The apparatus of C, wherein the pump housing includes an exhaust valve disposed at the distal end of the pump housing, the exhaust valve being configured to allow air to flow from within the pump housing to an exterior of the pump housing.

E. The apparatus of D, further comprising a biasing member configured to apply a distal force to the piston; and wherein the piston is movable distally within the pump housing responsive to the distal force applied by the biasing member, the intake valve being configured to close and the exhaust valve being configured to open.

F. The apparatus of E, wherein motion of the handle toward the pump housing causes the piston to move proximately within the pump housing, the intake valve being configured to open and the exhaust valve being configured to close.

G. A system comprising: a medical device including: a handle; and an insertion portion; and an apparatus including: a pump housing having a distal end attachable to a medical device and a longitudinal axis; a piston slidably receivable within the pump housing, the piston being configured to create a seal between a distal portion of the pump housing and a proximal portion of the pump housing; and a handle rotatably attachable to the housing, the handle being configured to move in a direction different from the longitudinal axis of the pump housing.

H. The system of G, wherein the pump housing includes a pump housing, wherein the pump handle including: a handle; a handle arm; and a pin pivot passage disposed between the handle and the handle arm, wherein the apparatus further includes: a fitting disposed at a distal end of the pump housing, the fitting attachable to the handle of the medical device; a piston rod couplable to a proximal side of the piston; a bracket mounted to an external surface of the pump housing at a proximal end of the pump housing; and a pivot pin couplable to the bracket and received within the pin pivot passage of the pump handle.

I. The system of H, wherein the pump housing includes an intake valve disposed between the fitting and a distal end of the pump housing, the intake valve being configured to allow air to flow proximately between the distal end of the pump housing and the pump housing.

J. The system of I, wherein the pump housing includes an exhaust valve disposed at the distal end of the pump housing, the exhaust valve being configured to allow air to flow from within the pump housing to an exterior of the pump housing.

K. The system of J, wherein motion of the handle away from the pump housing causes the piston to move distally within the pump housing, the intake valve being configured to close and the exhaust valve being configured to open.

L. The system of J, wherein motion of the handle toward the pump housing causes the piston to move proximately within the pump housing, the intake valve being configured to open and the exhaust valve being configured to close.

M. The system of G, wherein the medical device includes a needle, the handle including a stylet inlet port couplable to the fitting.

N. The system of M, wherein the fitting and the stylet inlet port include Luer fittings.

O. The system of G, wherein the system further comprises: a multi-lumen catheter device including: a flexible insertion tube comprising two or more lumen; and a handle being configured to be couplable to an endoscope, the handle including: a base section; and an inlet port being configured to be slidably received longitudinally along the base section; and a needle, wherein the handle of the medical device includes a stylet inlet port couplable to the distal end of the pump housing.

P. The system of O, wherein the distal end of the pump housing and the stylet inlet port include Luer fittings.

Q. A method comprising: placing a hand pump in a pre-activated state, the hand pump including: a piston; a piston housing configured to receive the piston therein; a fitting disposed at a distal end of the piston housing; and a handle configured to move the piston within the piston housing; and placing the hand pump in the activated state responsive to a distal end of a medical device being disposed adjacent to a target tissue and the fitting being coupled to the medical device.

R. The method of Q, wherein; the piston is in a distal position within the piston housing as a result of the hand pump being in the pre-activated state; and the piston is moving in a proximal direction within the piston housing as a result of the hand pump being in the activated state.

S. The method of R, further comprising: responsive to the piston moving in a distal direction within the piston housing, expelling air from the piston housing through a first one-way valve and blocking air from entering the piston housing via a second one-way valve located between the fitting and the piston housing.

T. The method of S, further comprising: responsive to the piston moving in a proximal direction within the piston housing, receiving air into the piston housing via the second one-way valve located between the fitting and the piston housing and blocking air from exiting the first one-way valve.

In some instances, one or more components may be referred to herein as “configured to,” “configured by,” “configurable to,” “operable/operative to,” “adapted/adaptable,” “able to,” “conformable/conformed to,” etc. Those skilled in the art will recognize that such terms (for example “configured to”) generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (for example, “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (for example, the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

While the disclosed subject matter has been described in terms of illustrative embodiments, it will be understood by those skilled in the art that various modifications can be made thereto without departing from the scope of the claimed subject matter as set forth in the claims.

What is claimed is:

1. An apparatus comprising:
a pump housing having a distal end attachable to a medical device and a longitudinal axis;
a piston slidably receivable within the pump housing, the piston being configured to create a seal between a distal portion of the pump housing and a proximal portion of the pump housing; and
a handle rotatably attachable to the housing and the piston, the handle including a portion being configured to move in a direction different from the longitudinal axis of the pump housing.

2. The apparatus of claim 1, wherein:
the pump handle includes:
a handle;
a handle arm; and
a pin pivot passage disposed between the handle and the handle arm; and
the apparatus further comprises:
a fitting disposed at a distal end of the pump housing, the fitting attachable to a handle of a medical device;
a piston rod couplable to a proximal side of the piston;
a bracket mounted to an external surface of the pump housing at a proximal end of the pump housing; and
a pivot pin couplable to the bracket and received within the pin pivot passage of the pump handle.

3. The apparatus of claim 2, wherein the pump housing includes an intake valve disposed between the fitting and a distal end of the pump housing, the intake valve being configured to allow air to flow proximately between the distal end of the pump housing and the pump housing.

4. The apparatus of claim 3, wherein the pump housing includes an exhaust valve disposed at the distal end of the pump housing, the exhaust valve being configured to allow air to flow from within the pump housing to an exterior of the pump housing.

5. The apparatus of claim 4, further comprising a biasing member configured to apply a distal force to the piston; and
wherein the piston is movable distally within the pump housing responsive to the distal force applied by the biasing member, the intake valve being configured to close and the exhaust valve being configured to open.

6. The apparatus of claim 5, wherein the piston is movable proximately within the pump housing responsive to motion of the handle toward the pump housing, the intake valve being configured to open and the exhaust valve being configured to close.

7. A system comprising:
a medical device including:
a handle; and
an insertion portion; and
an apparatus including:
a pump housing having a distal end attachable to the medical device and a longitudinal axis;
a piston slidably receivable within the pump housing, the piston being configured to create a seal between a distal portion of the pump housing and a proximal portion of the pump housing; and
a handle rotatably attachable to the housing, the handle being configured to move in a direction different from the longitudinal axis of the pump housing.

8. The system of claim 7, wherein:
the pump housing includes a pump housing;
the pump handle includes:
a handle;
a handle arm; and
a pin pivot passage disposed between the handle and the handle arm; and
the apparatus further includes:
a fitting disposed at a distal end of the pump housing, the fitting attachable to the handle of the medical device;
a piston rod couplable to a proximal side of the piston;
a bracket mounted to an external surface of the pump housing at a proximal end of the pump housing; and
a pivot pin couplable to the bracket and received within the pin pivot passage of the pump handle.

9. The system of claim 8, wherein the pump housing includes an intake valve disposed between the fitting and a distal end of the pump housing, the intake valve being configured to allow air to flow proximately between the distal end of the pump housing and the pump housing.

10. The system of claim 9, wherein the pump housing includes an exhaust valve disposed at the distal end of the pump housing, the exhaust valve being configured to allow air to flow from within the pump housing to an exterior of the pump housing.

11. The system of claim 10, wherein the piston is movable distally within the pump housing responsive to motion of the handle away from the pump housing, the intake valve being configured to close and the exhaust valve being configured to open.

12. The system of claim 10, wherein the piston is movable proximately within the pump housing responsive to motion of the handle toward the pump housing, the intake valve being configured to open and the exhaust valve being configured to close.

13. The system of claim 7, wherein the medical device includes a needle, the handle including a stylet inlet port couplable to the fitting.

14. The system of claim 13, wherein the fitting and the stylet inlet port include Luer fittings.

15. The system of claim 7, further comprising:
a multi-lumen catheter device including:
a flexible insertion tube comprising two or more lumen;
a handle configured to be couplable to an endoscope, the handle including:
a base section; and
an inlet port being configured to be slidably received longitudinally along the base section and configured to slidably receive the insertion portion of the medical device; and
wherein:
the medical device includes a needle slidably received within the insertion portion; and
the handle of the medical device includes a stylet inlet port couplable to the distal end of the pump housing.

16. The system of claim 15, wherein the distal end of the pump housing and the stylet inlet port include Luer fittings.

17. A method comprising:
placing a hand pump in a pre-activated state, the hand pump including:
a piston;
a piston housing configured to receive the piston therein;
a fitting disposed at a distal end of the piston housing; and
a handle configured to move the piston within the piston housing; and
placing the hand pump in the activated state responsive to a distal end of a medical device being disposed adjacent to a target tissue and the fitting being coupled to the medical device.

18. The method of claim 17, wherein;
the piston is in a distal position within the piston housing as a result of the hand pump being in the pre-activated state; and
the piston moves in a proximal direction within the piston housing as a result of the hand pump being in the activated state.

19. The method of claim 18, further comprising:
responsive to the piston moving in a distal direction within the piston housing, expelling air from the piston housing through a first one-way valve and blocking air from entering the piston housing via a second one-way valve located between the fitting and the piston housing.

20. The method of claim 19, further comprising:
responsive to the piston moving in a proximal direction within the piston housing, receiving air into the piston housing via the second one-way valve located between the fitting and the piston housing and blocking air from exiting the first one-way valve.
